(19) United States
(12) Patent Application Publication
(10) Pub. No.: US 2009/0228084 A1
(43) Pub. Date: Sep. 10, 2009

(54) Method of non invasive stimulation of acupuncture or acupressure points utilizing at least one electrostatic field

(76) Inventor: Hung Chuang Chen, Fremont, CA (US)

Correspondence Address: Hung Chuang Chen, 43194 Nielsen Court, Fremont, CA, 94539, US

(21) Appl. No.: 12/045,597

(22) Filed: Mar. 10, 2008

(51) Int. Cl. A61N 1/36

(52) U.S. Cl. 607/115

(57) ABSTRACT

A non invasive method of stimulating nerves and/or acupuncture/acupressure point(s) using at least one electrostatic field, whereby the said electrostatic field(s) is generated by a generator, said generator is a battery(ies) or a pair of charged plates. The generator voltage is between 1.2 and 100 volts. The electrostatic field generator(s) may be separated from an electrostatic field releasing window(s) by a filter(s), which electrostatic field window(s) is(are) placed on a skin contact pertaining to an acupuncture/acupressure point(s). The method will not release any current to the human skin. This method of stimulation is safer, more efficient and comfortable than its competitive technology, as the patients will not suffer from any discomfort caused by a current. This method of stimulation may be used in controlling certain symptoms and diseases in humans (e.g. nausea, insomnia, stress, hypertension) in accordance with Eastern medicine principles.

### CROSS-REFERENCES TO RELATED APPLICATIONS

U.S. patent documents

| D274557 | July 1984 | Griffith |
|---|---|---|
| D356433 | March 1995 | Humphrey |
| 4981146 | January 1991 | Bertolucci |
| 5078728 | January 1992 | Giarratano |
| 5695520 | December 1997 | Bruckner et al. |
| 5709647 | January 1998 | Ferber |
| 5774424 | June 1998 | Yoo |
| 5601598 | February 1997 | Fisher |
| 6228103 | May 2001 | Grey et al |

### STATEMENT REGARDING FEDERALLY SPONSORED RESEARCH OR DEVELOPMENT

Not Applicable

### The Names of the Parties to a Joint Research or Development

Not Applicable

### Incorporation-By-Reference of Material Submitted on a Compact Disc

Not Applicable

### BACKGROUND OF THE INVENTION

1. Field of the Invention

The methods and devices described relate generally to the fields of acupuncture, acupressure and non-invasive stimulation of nerves.

2. Background Art

The present invention relates to the area of treatment of various diseases related to human beings. Particularly, the present invention is related to non invasive methods of stimulating nerves using electrostatic field(s) according to the principles of acupressure and acupuncture. This method of stimulation may be used in controlling certain symptoms and diseases in humans (e.g. nausea, insomnia, stress, hypertension) in accordance with Eastern medicine principles. The invention is also related to the embodying stimulation devices suitable for use by patients with minimum supervision of a medical caregiver. Traditional acupressure involves the stimulation of acupressure point(s) through pressure, be it of human contact (such as fingers of a practitioner of the art, fists, elbows, etc) or through the intermediate of objects. Inventors have applied this method to design devices which deliver the pressure without the need for the human hand e.g., Giarratano, U.S. Pat. No. 5,078,728, Humphrey, U.S. Pat. No. D356,433, Bruckner et al., U.S. Pat. No. 5,695,520, Griffith, U.S. Pat. No. D274,557, Ferber, U.S. Pat. No. 5,709,647. Yoo (U.S. Pat. No. 5,774,424). Although proven effective, the benefits of acupressure have been shown to wear out over time, as the body becomes accustomed to constant pressure. Different inventors have aimed at solving the problem by changing the pressure: Fisher (U.S. Pat. No. 5,601,598) describes a method in which the user is required to periodically stretch a band to cause changing pressure, making the user stop using the band in order to accomplish the result of changing pressure; Grey et al (U.S. Pat. No. 6,228,103) uses a small DC motor to rotate a cam across the acupressure module to prevent the nerve from accommodation over a small predetermined period of time. While some of these methods circumvent the constancy over a short period of time, their efficacy is limited over time and their use may cause discomfort for the patients, either because they require an active participation of the patient, or because the change in pressure causes tactile discomfort, sometimes even “prickling” sensations. Anti-nausea patients, as an example of users, range from travelers on a cruise to terminally ill patients—and the patients requiring treatment over extended periods of time need not only ease of use but also need to avoid discomfort in the form of interruption in their activities or in the form of physical, sensorial discomfort.

Traditional acupuncture, as now practiced, involves the insertion of stainless steel needles through the patient's skin, into various body areas. A low-frequency current may be applied to the needles to produce greater stimulation. Treatment is applied to “acupuncture point(s)” which are said to be located throughout the body in accordance with Eastern medicine principles. In the last couple of decades, the TENS (Transcutaneous Electric Nerve Stimulation) was invented, which substitutes the needles for electrodes; however, the device still applies current to human's “acupuncture point(s)”. These principles have been applied to portable devices, eg. Bertolucci, Nausea Control Device, U.S. Pat. No. 4,981,146 (Jan. 1, 1991) describes a nausea control device in the form of a watch-like housing attachable to the human wrist by an adjustable attachment band which applies non-invasive nerve stimulation to the inside of the wrist (palm side) to a point called P6 in the art of acupuncture. This non-invasive stimulation is achieved by applying electricity as passed by two electrodes. This method of stimulation, although non-invasive, can present discomfort for the user, as the sensitivity to the electricity differs according to the user and the longevity of their use, including the accommodation to the current level. Referred pain may also incur in certain areas, such as tingling or prickling sensations in the hand for TENS used in the wrist. In addition, the device requires the constant use of gel or electrodes, which may present with dermatological issues in certain patients, as well as a monetary and time inconvenience caused by having to replace electrodes on a regular basis.

Attempts have been made to circumvent both the constant pressure problem and the discomfort level by offering different levels of acupressure and/or electro-acupuncture. These attempts for acupressure require a relatively short period of usage, after which the nerve accommodation issue reoccurs, and the electro-acupuncture may reach a higher level of accommodation, as the changing current numbs the nerve.

The present invention(s) applies to the body of acupuncture and acupressure point(s) covered by both methods as one ensemble, and aims at providing both short-term and long-term patients the relief provided by stimulating these acupuncture/acupressure point(s) during normal activities with minimal supervision from a medical caregiver, such nausea control patients or other categories of patients (comprising stress relief and hypertension patients).

### BRIEF SUMMARY OF THE INVENTION

This section is for the purpose of summarizing some aspects of the present invention(s) and to briefly introduce some preferred embodiments. Simplifications or omissions in this section as well as in the abstract and the title may be made to avoid obscuring the purpose of this section, the abstract and the title. Such simplifications or omissions are not intended to limit the scope of the present invention(s).

Instead of using stainless steel needles, or apply currents to human's skin, the current invention(s) pertains to a method using an apparatus for providing non-invasive stimulation on the acupressure/acupuncture point(s) by directing a filtered electrostatic field to the acupressure/acupuncture point(s). This method of stimulating the acupuncture/acupressure point(s) aims at eliminating the discomfort and pain caused by needles or electric fields used in TENS as well as making the stimulation possible by the patient autonomously. Several techniques of using filtered electrostatic field(s) generator(s) and their applications are disclosed.

According to one aspect of the present invention, an applied embodiment of the method disclosed comprises a housing and may enclose some or all of the following to form a non-invasive medical device: filter(s), filter protector(s), circuit board(s), battery(ies), transformer(s), transformer connector(s), and a pair(s) of charged plates. The device may comprise a strip(s), clip(s) or tape(s) to fix a releasing window(s) firmly placed over the acupressure/acupuncture point(s). The described device may comprise at least one pair of charged plates used to release an electrostatic field(s). The charged plates receive power from a battery, batteries, a transformer or a power outlet. When such electrostatic field(s) passes a corresponding filter, it forms a filtered electrostatic field(s) released through a corresponding window, which is placed on a skin contact pertaining to an acupressure/acupuncture point(s).

According to another aspect of the present invention, a composition comprises many treatment heads to form a single user or multi-user medical device which is able to stimulate a plurality of acupressure/acupuncture point(s) of one or more person(s) simultaneously. Each treatment head may comprise a strip(s), clip(s) or tape(s) used to place and hold the treatment head on a skin contact pertaining to an acupressure/acupuncture point(s). The treatment head comprises at lease one pair of charged plates used to release an electrostatic field(s). When such electrostatic field(s) passes through a corresponding filter, it forms a filtered electrostatic field(s) released through a corresponding window which is placed on a skin contact pertaining to an acupressure/acupuncture point(s). Each pair of charged plates and its filter can be enclosed in a plastic housing (the treatment head) with prolonged cables connected to a control unit to receive both the electrical power and the treatment instructions. One of the embodiments comprises many treatment heads to form a single user or multi-user medical device which is able to stimulate a plurality of acupressure/acupuncture point(s) of one or more person(s) simultaneously. Each treatment head may comprise a strip(s), clip(s) or tape(s) to fix the treatment head firmly placed on a skin contact pertaining to an acupressure/acupuncture point(s).

According to another embodiment of the present invention, the electrostatic field(s) is(are) generated by battery(ies) or rechargeable battery(ies) which may be recharged. A housing may enclose some or all of the following to form a non-invasive medical device: the electrostatic field generating battery(ies), the filter(s), the filter protector(s), a circuit board and the power source battery(ies) or power connector. The device may comprise a strip(s), clip(s) of tape(s) used to place and hold the treatment head firmly placed on a skin contact pertaining to an acupressure/acupuncture point(s).

Other objects, features, advantages, benefits of the invention will become more apparent from the following detailed description of a preferred embodiment, which proceeds with reference to the accompanying drawings.

### BRIEF DESCRIPTION OF THE SEVERAL VIEWS OF THE DRAWINGS

The present invention will be readily understood by the following detailed descriptions in conjunction with the accompanying drawings, wherein like reference numerals designate like structural elements, and in which:

FIG. 01A to FIG. 01B show two exemplary embodiments of Electrostatic Field Generator 01. It generates electrostatic field(s) through a pair of charged plates (FIG. 01A) or through a battery (FIG. 01B)

FIGS. 01C and 01D show the other two exemplary embodiments of Electrostatic Field Generator 01 with a filter protector mechanism, consisting of a Protector 21 and an Axis 22. It generates electrostatic field(s) through a pair of charged plates (FIG. 01C) or through a battery (FIG. 01D)

FIG. 01E shows an explosion table of the elements in FIGS. 01A, 01B, 01C and 01D. It shows their top view in the 2nd column and side view in the 3rd column.

FIGS. 01F, 01G and 01H show how an exemplary embodiment of the Filter Protector 21 moves to protect the Filter 02.

FIG. 02 shows a cross-sectional view of an exemplary Filter 02 of an Electrostatic Field Generator 01.

FIG. 03A shows an exemplary embodiment of a medical Device 03 with an Electrostatic Field Generator 01 using a Field Generating Battery 18.

FIG. 03B and FIG. 03C show two exemplary embodiments of a Device 03 with an Electrostatic Field Generator 01 using Charged Plates 11 powered by a Powered Battery 17 or a Power Connector 19 respectively.

FIG. 03D to FIG. 03G show 4 exemplary embodiments of a Device 03 wherein the Electrostatic Field Generator 01 is powered by Powered Battery(ies) 17 (FIG. 03D and FIG. 03F) or by a Power Connector 19 (FIG. 03E and FIG. 03G) and wherein the Device 03 comprises one (FIG. 03D and FIG. 03E) or more (FIG. 03F and FIG. 03G) Electrostatic Field Generators 01 for one or more acupressure/acupuncture point(s).

FIG. 03H shows an exemplary way to apply the device to the human skin by using an exemplary watch-like belt to be worn on the wrist with its Electrostatic Field Releasing Window(s) 13 firmly placed on a skin contact pertaining to an acupressure/acupuncture point(s) 14. An exemplary acupressure/acupuncture point P6 or Neiguan is used on this example in accordance with Eastern traditional medicine.

FIG. 04A shows a functional block diagram of a Circuit Board 04 on one exemplary embodiment of the present invention.

FIG. 04B and FIG. 04C show a flowchart or process of stimulating one or more acupuncture/acupressure points on a human in accordance with one embodiment of the present invention utilizing different Electrostatic Field Generators 01: Electrostatic Field Generator 01A, 01C or Electrostatic Field Generator 01 B, 01D respectively.

FIG. 05A shows an exemplary embodiment of a detached Treatment Head 05 with an Electrostatic Field Generator 01A which comprises a pair of Charged Plates 11 to generate an electrostatic field(s).

FIG. 05B shows an exemplary way to apply a Treatment Head 05 to the human skin by using a exemplary watch-like belt to be worn with its Electrostatic Field Releasing Window(s) 13 firmly placed on a skin contact pertaining to an acupressure/acupuncture point(s). An exemplary acupressure/acupuncture point(s) P6 or Neiguan is used on this example to treat certain diseases in accordance with Eastern traditional medicine.

FIG. 06 shows an exemplary embodiment of a device with more than one detached Treatment Head installed.

### DETAILED DESCRIPTION OF THE PREFERRED EMBODIMENTS OF THE INVENTION

The present invention pertains to non invasive methods of stimulation of acupuncture or acupressure points for relieving symptoms of certain diseases such as nausea relief, stress relief, controlling blood pressure, etc. The present invention discovers that directing at least one electrostatic field over the acupuncture/acupressure points delivers the same therapeutic effects of acupuncture treatment, without having to use acupuncture needles, and without having to puncture skin to reach acupuncture points as practiced in conventional acupuncture therapies; it also delivers the same therapeutic effects of acupressure treatment without have to use the hands as practiced in conventional acupressure therapies.

The detailed description of the invention is presented largely in terms of procedures, steps, logic blocks, processing, and other symbolic representations that directly or indirectly resemble the operations of data processing devices. These process descriptions and representations are typically used by those skilled in the art to most effectively convey the substance of their work to others skilled in the art.

Reference herein to “one embodiment” or “an embodiment” means that a particular feature, structure, or characteristic described in connection with the embodiment can be comprised in at least one embodiment of the invention. The appearances of the phrase “in one embodiment” in various places in the specification are not necessarily all referring to the same embodiment, nor are separate or alternative embodiments mutually exclusive of other embodiments. Further, the order of blocks in process flowcharts or diagrams representing one or more embodiments of the invention do not inherently indicate any particular order nor imply any limitations in the invention.

According to one aspect of the present invention, a preferred embodiment comprises a watch-like housing used to encase at least an electrostatic field generator which may comprise any or all of the following: charged plates, battery(ies), rechargeable battery (ies), electrostatic field filter(s), electrostatic field releasing window(s). When such an electrostatic field(s) goes through a filter, the current invention directs the filtered electrostatic field(s) to the human skin through a releasing window contact to stimulate an acupressure/acupuncture point(s). The voltage of the charged plates is at a prescribed voltage of approximately 1.2 to 100 V. Various power sources such as a battery(ies), battery box, transformer, direct power outlet may be attached to the device to achieve various forms of devices.

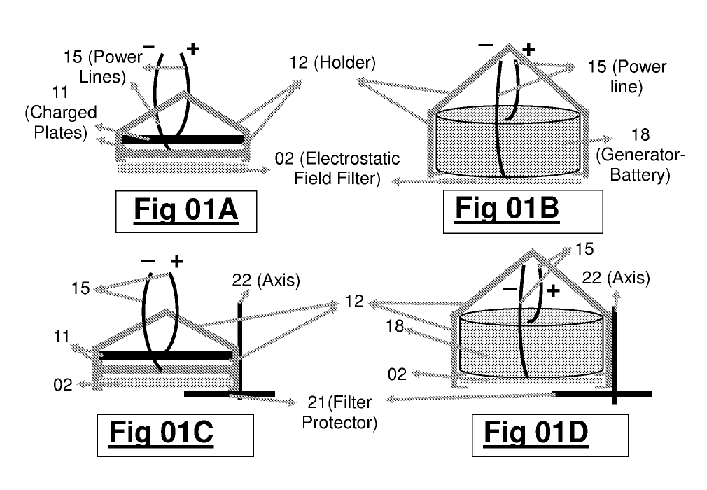

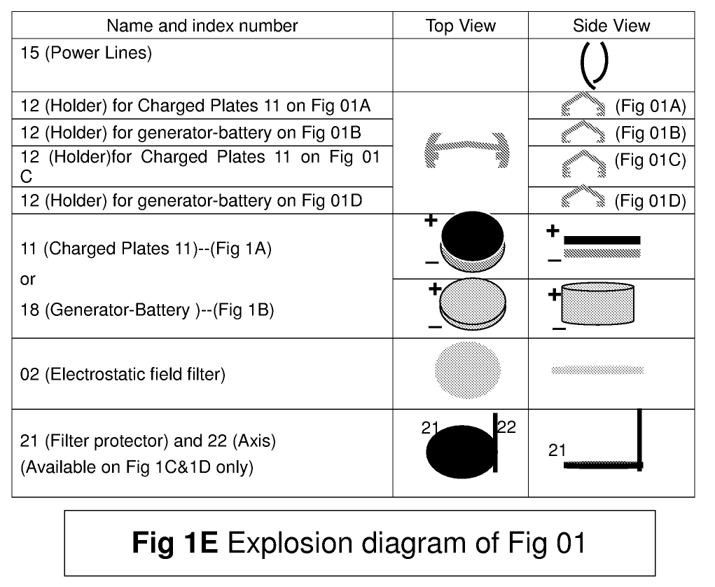

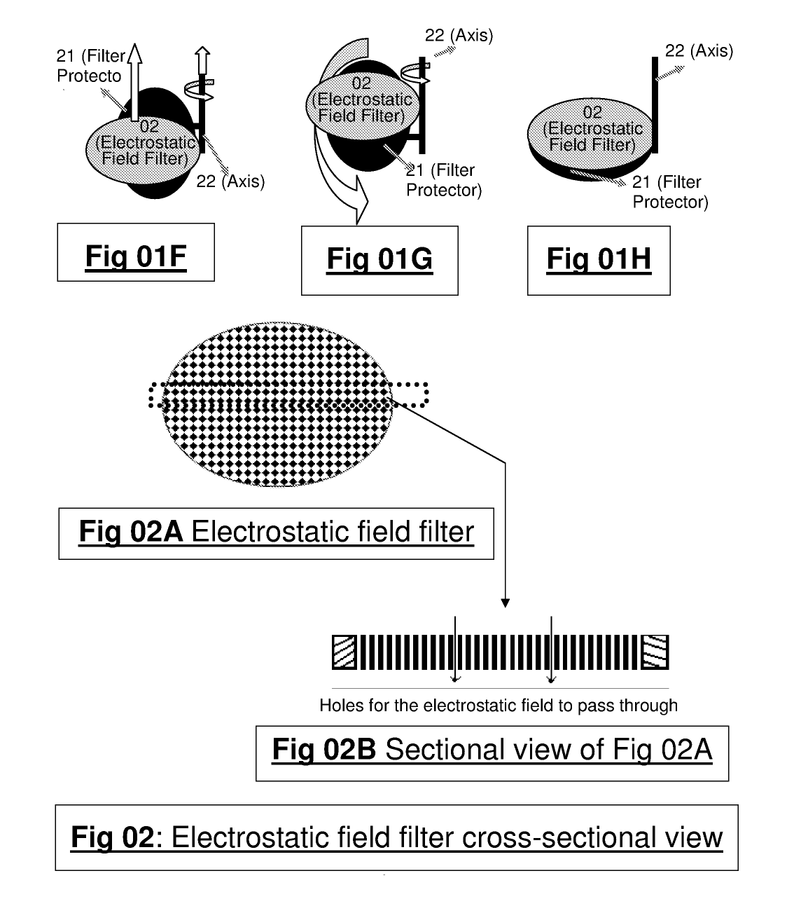

Referring now to the drawings, in which like numerals refer to like parts throughout the several views. From FIG. 01A to FIG. 01D shows 4 preferred designs of an Electrostatic Field Generator 01. Wherein, FIG. 01A shows the Electrostatic Field Generator 01 uses a pair of Charged Plates 11 held by a Holder 12. An Explosion diagram is shown on FIG. 01E. The Charged Plates 11 are charged through the Power Line 15 and generate electrostatic field(s) which passes the Filter 02 and forms a filtered electrostatic field(s) which stimulates acupuncture/acupressure points in accordance with Eastern medicine principles. Alternatively, the Charged Plates 11 can be replaced with a Field Generating Battery 18 as shown on FIG. 01B.

FIG. 01C shows an Electrostatic Field Generator 01 comprising a pair of Charged Plates 11 which are charged by the Power Line 15. A Holder 12 holds both the Charged Plates 11 and the Filter 02. An Axis 22 is joined to the Holder 12 and a Filter Protector 21 is further joined to the Axis 22. An Explosion diagram is shown on FIG. 01E. The Electrostatic Field Generator 01 generates the filtered electrostatic field(s) when the electrostatic field(s) passes through the Filter 02. (I will describe how the Filter Protector 21 and Axis 22 function to protect the Filter 02 later on FIGS. 01F, 01G and 01H). Alternatively, the Charged Plates 11 can be replaced with a Field Generating Battery 18 as shown on FIG. 01B.

FIG. 01E shows an Explosion table of FIGS. 01A, 01B, 01C and 01D. It shows a top view in the 2nd column and a side view of the items described in the 3rd column. There are four shapes of holders: the first one with two notches to hold a pair of Charged Plates 11, as used on FIG. 01A; the second one, with only one notch to hold a Field Generating Battery 18, as shown on FIG. 01B; the third one, with three notches to hold a pair of Charged Plates 11 and the Filter 02, as used on FIG. 01C; and the last one comprises 2 notches to hold the Field Generating Battery 18 and the Filter 02, as used on FIG. 01D. The last row on the FIG. 01E contains the Filter Protector 21 and Axis 22; they are used only on FIG. 01C and FIG. 01D.

FIGS. 01F, 01G and 01H show how an exemplary embodiment of Filter Protector 21 works. After the Electrostatic Field Generator 01 has been used, the Axis 22 will spin to promote the Holder 12 shown on FIG. 01F and FIG. 01G and make room for Filter Protector 21 to move in. The Axis 22 spins further and causes the Filter Protector 21 to rotate and move into right below the Filter 02 shown on FIG. 01H to protect the Filter 02 against any possible dust coming from the Electrostatic Field Releasing Window(s) 13. When the Electrostatic Field Generator 01 begins to be used, the Axis 22 spins reversely to move the Filter Protector 21 away from the Filter 02 and further spinning will lower the Holder 12 and move the Electrostatic Field Generator 01 to the ready-to-use position.

FIG. 02 shows a side view of a magnified Filter 02. There are hundred of holes on the filter. When the electrostatic field(s) passes through the holes in the filter, it generates a filtered electrostatic field(s). The current invention is to apply the filtered electrostatic field(s) to a human skin contact pertaining to an acupressure/acupuncture point(s).

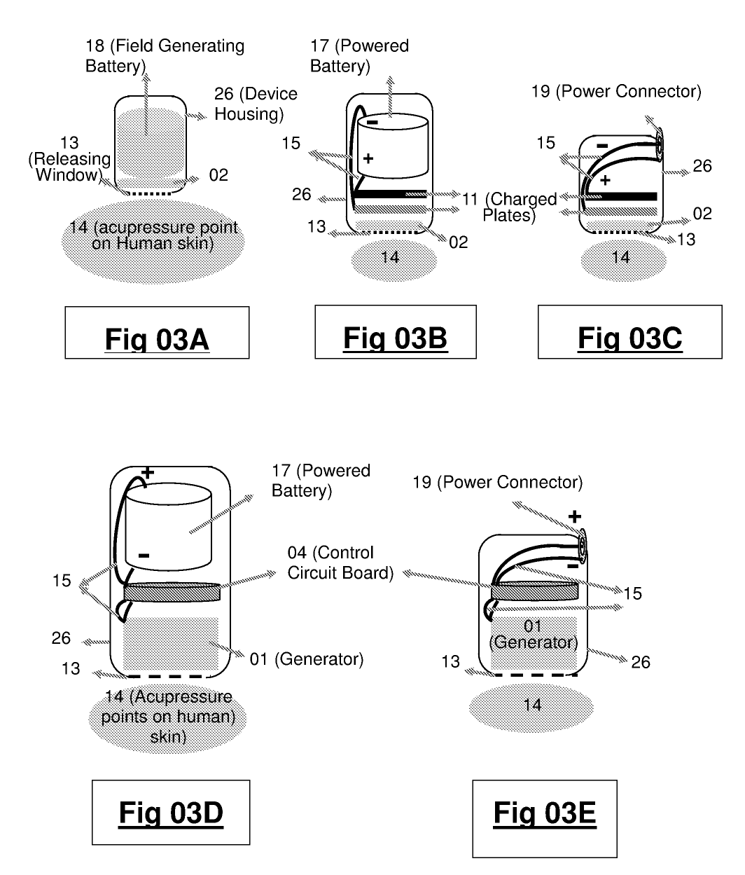

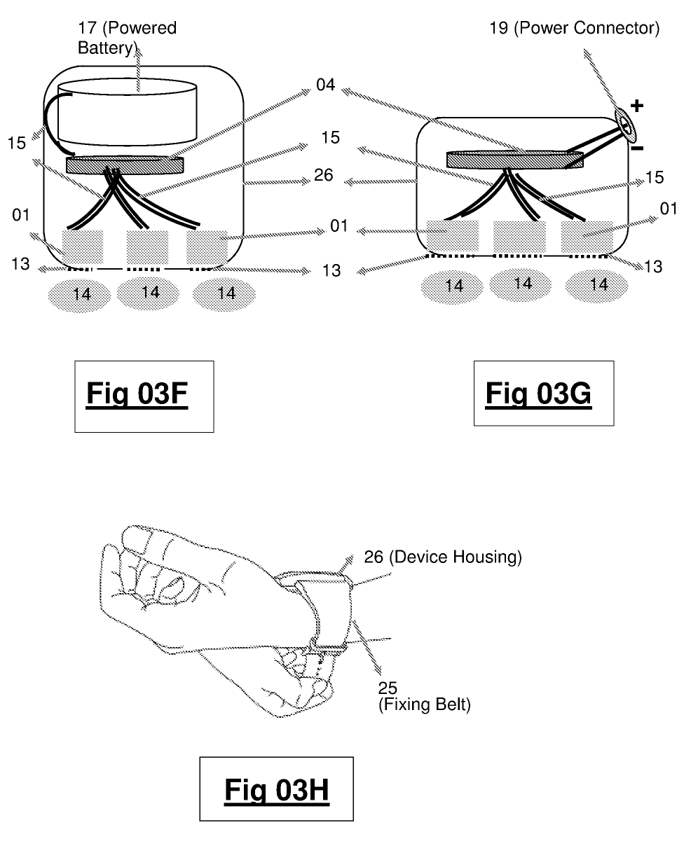

FIG. 03A to FIG. 03G shows 7 preferred embodiments of the invention, which is a completely non-invasive device (device 03) and may be used by a patient with or without supervision from a caregiver or from a hospital facility. Optionally, the device 03 may comprise an Output Unit 23 (such as LED or LCD) (not shown) and/or an Input Unit 24 (such as a keypad) (not shown) to communicate with the user.

According to one embodiment shown on FIG. 03A, the Device 03 uses a single Field Generating Battery 18 and a Filter 02 to generate the filtered electrostatic field(s) to be released through the Electrostatic Field Releasing Window(s) 13 which is placed on a skin contact pertaining to an acupressure/acupuncture point(s) 14.

According to another embodiment shown on FIG. 03B, the Device 03 uses a Powered Battery 17 to charge the Charged Plates 11 and a Filter 02 to generate the filtered electrostatic field(s) to be released through the Electrostatic Field Releasing Window(s) 13 which is placed on a skin contact pertaining to an acupressure/acupuncture point(s) 14.

According to another embodiment shown on FIG. 03C, the Device 03 uses a Power connector 19 to receive external power to replace the Powered Battery(ies) 17 shown on FIG. 03B.

According to 4 preferred embodiments shown on FIGS. 03D, 03E, 03F and 03G, the Device 03 is equipped with a Circuit Board 04 which is able to control the voltage of the Electrostatic Field Generator 01. It can control the polarity of the Charged Plates 11 on those Electrostatic Field Generator 01 equipped with Charged Plates 11. The Electrostatic Field Generator 01 releases the filtered electrostatic field(s) through the Electrostatic Field Releasing Window(s) 13 which is placed on a skin contact pertaining to an acupressure/acupuncture point(s) 14. The Circuit Board 04 may receive commands from users through Input Unit(s) 24 (such as buttons and/or key pads) not shown on the drawings, then process accordingly and send the status of the device to an Output Unit(s) 23 (such as LED or LCD) not shown on the drawings. The devices 03 shown on FIG. 03D and FIG. 03F are powered by a Powered Battery 17; they are equipped with one and three Electrostatic Field Generators 01 respectively. The Device 03 shown on FIG. 03E and FIG. 03G are powered by outside power sources through their Power Connector 19; they are equipped with one and three Electrostatic Field Generators 01 respectively.

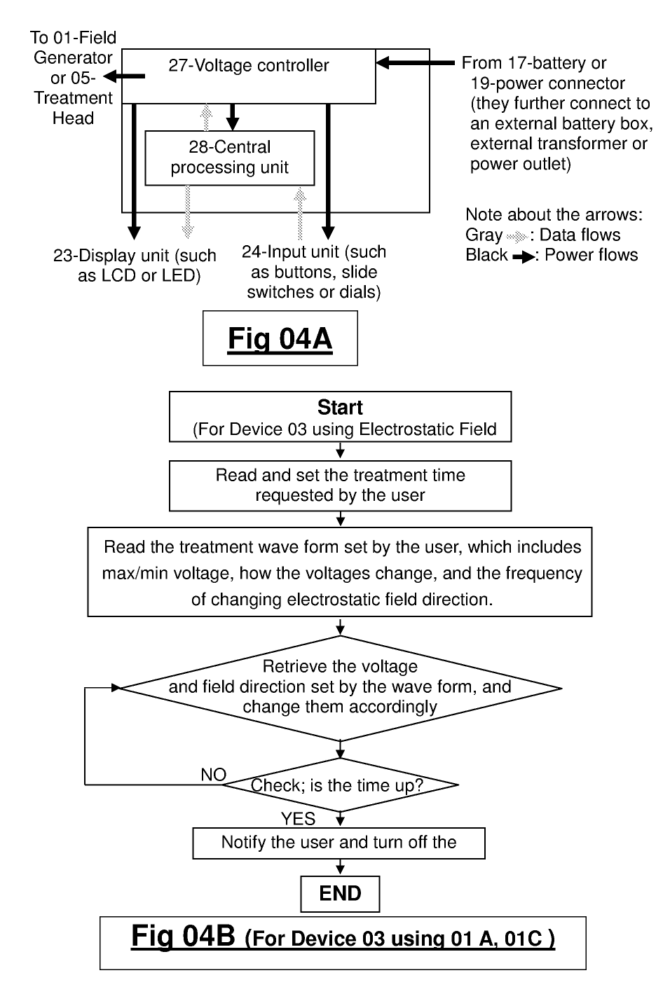

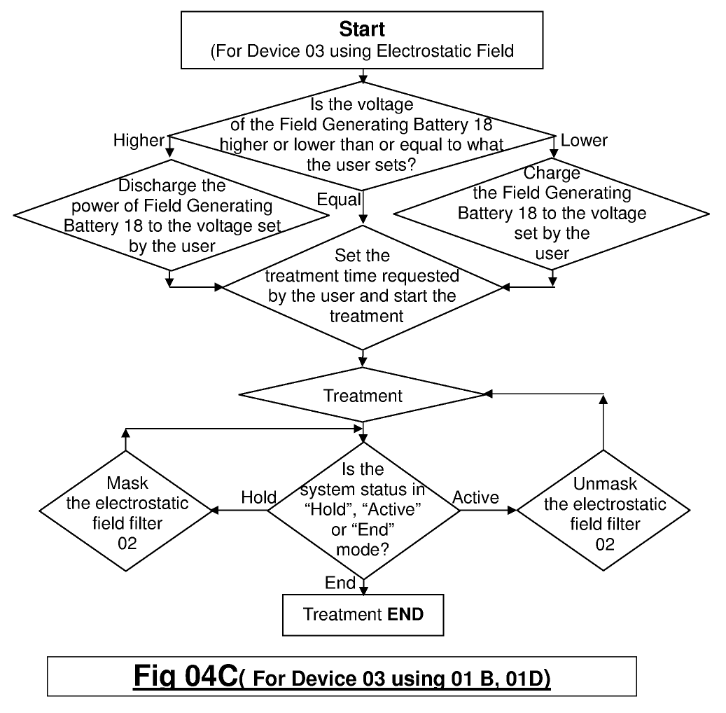

FIG. 04A shows an exemplary functional block diagram of a Circuit Board 04 on one exemplary embodiment of the present invention. The Circuit Board 04 is equipped with a Processing Center 28 which controls the Voltage Controller 27, which may be a transformer or a battery charger/discharger, and further controls the voltage of the Electrostatic Field Generator 01. The Processing Center 28 may also control the field direction of the Electrostatic Field Generator 01 A or 01C by changing the polarity of the Charged Plates 11. The Circuit Board 04 accepts user(s) commands through an Input Unit 24 and displays the system status onto an Output Unit 23 (such as speakers, LCD etc).

Referring now to the drawings FIG. 04B and FIG. 04C; they show an exemplary flowchart of a Circuit Board 04 on a Device 03 using the Charged Plates 11 Electrostatic Field Generator 01 shown on FIG. 01A and 01C. The system removes the Filter Protector 21 (if any) and reads the user's settings then confirming to the definition of the wave form set by the user by changing the voltage over time and the field direction of the Charged Plates 11 of the Electrostatic Field Generator 01 (FIG. 01A, FIG. 01C). When treatment time is due the system then restores the Filter Protector 21 and turns off the power.

FIG. 04C shows another exemplary flowchart of a Circuit Board 04 on a Device 03 using the Battery Electrostatic Field Generator 01B or 01D. The system removes the Filter Protector 21 (if any) and reads the user(s)'s settings and the current voltage of the rechargeable Field Generating Battery 18 then charges (discharges) the Field Generating Battery 18 to increase (decrease) the voltage accordingly. When treatment time is due or in hold status the system then restores the Filter Protector 21 and turns off the power.

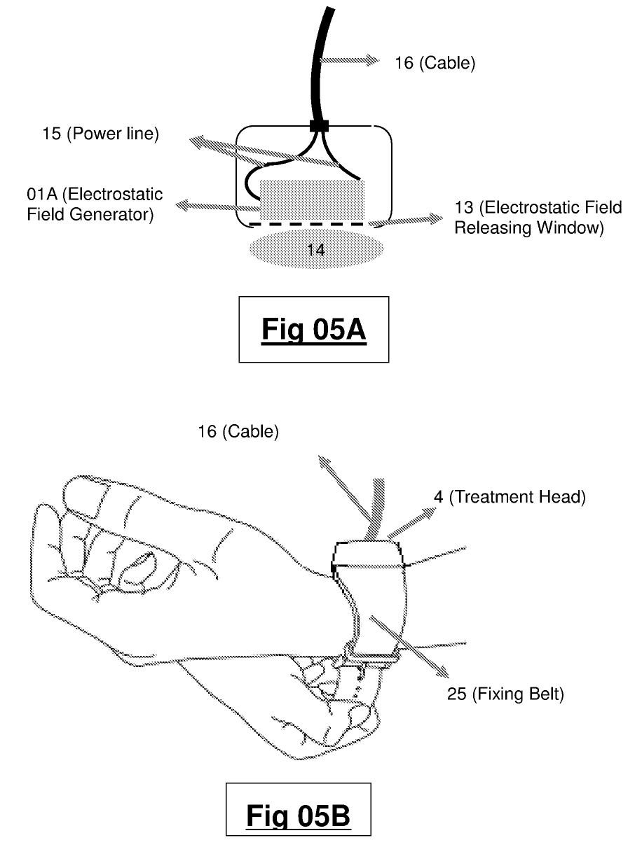

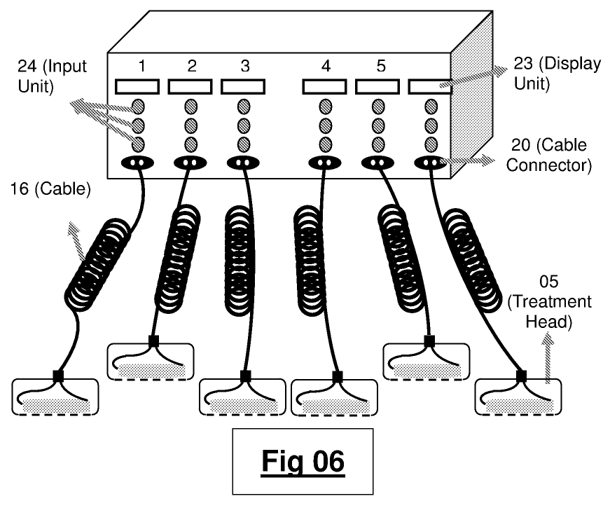

FIGS. 05A, 05B show a Treatment Head 05 with extended Cable 16. The Treatment Head 05 contains an Electrostatic Field Generator 01A which is controlled by a Circuit Board 04 (not shown) outside the Treatment Head 05 and inside a Treatment Machine 06 (see FIG. 06) through the cable. The Treatment Head may be fixed to a skin contact pertaining to an acupressure/acupuncture point(s) 14 by a belt as shown on FIG. 05B.

FIG. 06 shows a Treatment Machine 06, which is an exemplary application of the Treatment Head 05, and is comprised of a plurality of Treatment Heads connected via extended Cables 16. The Treatment Machine 06 is equipped with an Input unit 24, an Output Unit 23 and a Cable Connector 20 for each Treatment Head 05; so that each Treatment Head 05 may have its own settings and may operate differently and independently. The advantage of this application is that the Treatment Machine 06 can be used by multiple users simultaneously and can stimulate different acupressure/acupuncture point(s) of each user.

The present invention has been described in sufficient details with a certain degree of particularity. It is understood to those skilled in the art that the present disclosure of embodiments has been made by way of examples only and that numerous changes in the arrangement and combination of parts may be resorted without departing from the spirit and scope of the invention as claimed. While the embodiments discussed herein may appear to comprise some limitations as to the presentation of the information units, in terms of the format and arrangement, the invention has applicability well beyond such embodiment, which can be appreciated by those skilled in the art. Accordingly, the scope of the present invention is defined by the appended claims rather than the forgoing description of embodiments.

What is claimed is:

1. A method of stimulation of acupuncture or acupressure points, said method comprising the steps of: mounting a non-invasive nerve stimulation device over an acupuncture or acupressure point; generating at least one electrostatic field; and delivering the electrostatic field(s) to the acupuncture or acupressure point to the patient(s) for relief of various symptoms (eg. nausea relief, stress relief, pre-menstrual symptoms, controlling blood pressure, etc.).

2. A method of stimulation of a plurality of acupuncture or acupressure points, said method comprising the steps of: mounting a non-invasive nerve stimulation device over a plurality of acupuncture or acupressure points in at least one patient; generating at least one electrostatic field; and delivering the electrostatic field(s) to the acupuncture or acupressure points to the patient(s) for relief of various symptoms (eg. nausea relief, stress relief, pre-menstrual symptoms, controlling blood pressure, etc.).

3. The method of claims 1 and 2, wherein the plurality of ensembles of points includes a first ensemble related to acupuncture points, and a second ensemble related to acupressure points.

4. The method of claims 1 and 2 wherein said delivering at least one electrostatic field step includes providing at least one filter, which is an object less than 2 mm in thickness and comprises more than 20 holes per square inch to allow the electrostatic field(s) to pass through and form filtered electrostatic fields.

5. The method of claims 1 and 2 wherein said generating at least one electrostatic field step includes providing a generator of variable voltage, approximately between 1.2 and 100 volts.

6. The method of claims 1 and 2 wherein said generating at least one electrostatic field step includes providing a generator of constant voltage, approximately between 1.2 and 100 volts.

7. The method of claims 1 and 2 wherein said generating at least one electrostatic field step comprises using a circuit board and a transformer for variable voltage of the generator to form an electrostatic field wave form.

8. The method of claims 1 and 2 wherein said generating at least one electrostatic field step comprises using a transformer for controlling the voltage of the generator.

9. The method of claims 5 and 6, wherein the generator is comprised of at least one battery, which may be rechargeable.

10. The method of claims 5 and 6, wherein the generator is comprised of at least one pair of Charged Plates 11.

11. The method said in claim 10, further comprising a transformer for varying the polarity of the generator during a treatment to form an electrostatic field wave form.

12. The method said in claim 7, wherein the circuit board further receives power from a variety of forms of power sources such as an external or internal transformer(s), battery(batteries) and/or power outlet(s).

13. The method said in claim 7, wherein the circuit board further receives commands from a variety of input units such as keypad, buttons, dials, slide switches or voice devices and may output the system information to a speaker or display units such as LCD or LED.
